(19) United States
(12) Patent Application Publication
(10) Pub. No.: US 2023/0172507 A1
(43) Pub. Date: Jun. 8, 2023

(54) Method, System and Apparatus for Investigating or Assessing Eye or Pupil Movement

(71) Applicant: Neurotologix Pty Ltd, Shenton Park (AU)

(72) Inventors: Katherine Ann Lewkowski, Shenton Park (AU); Gunesh P. Rajan, Luzerner (CH); Robert John Deacon, Bayswater (AU)

(21) Appl. No.: 17/922,196

(22) PCT Filed: Apr. 30, 2021
(86) PCT No.: PCT/AU2021/050398

§ 371 Date: Oct. 28, 2022

(30) Foreign Application Priority Data
Apr. 30, 2020 (AU) 2020901376

(51) Int. Cl. A61B 5/16; A61B 5/00; A61B 3/11

(52) U.S. Cl. CPC A61B 5/163; A61B 5/4023; A61B 3/11; A61B 5/0082; A61B 5/4863; G06T 2207/30041

(57) ABSTRACT

Method, system and apparatus (10) for capturing eye/pupil movement(s). Wearable apparatus (10) includes image capture means (12,18), a light source (16) to illuminate the eye(s). Video recordings can be stored in memory (20) and/or transmitted to a remote location. The apparatus (10) can include on-board image processing. Pupil shape and/or position can be determined e.g. from glints (28). Direction and speed of eye motion can be determined. Head movement (50) and eye motion (52) measurements can be obtained. Measurement by head movement sensors (32), body sensors (34) and/or environment sensors (38) (e.g. gravity) can be factored in to an assessment of the user's condition.

### FIELD OF THE INVENTION

The present invention relates to the investigation or assessment of changes in the eye, such as pupil position, pupil size, eye position and/or eye movement. Such investigation can include measuring and monitoring eye/pupil movement.

One or more forms of the present invention relate(s) particularly, though not exclusively, to an apparatus, system and/or method for the investigation of motion and/or changes in the eye and pupil, such as eye position, pupil size, pupil position and/or eye movement (such eye motion patterns as in nystagmus), which may be associated with vestibular and other neurological disorders.

### BACKGROUND TO THE INVENTION

The presence of a number of medical and health problems can be detected by changes in the eye and/or its pupil, including changes in the position, movement and dilation of the eye and/or its pupil.

For example, it has been found that one of the leading causes of dizziness and balance problems is associated with the vestibular system in the inner ear or in the vestibular pathways and control centre of central nervous system.

The presence of nystagmus during a dizziness attack can suggest to a clinician that there is a vertiginous component to it and the direction of the nystagmus may provide some evidence to a specialist in the field, of more specific information, such as which ear (or which part of the ear) has the active disease.

In addition, the pattern of the nystagmus of one or both eyes can also indicate which part of the central nervous system is affected.

Nystagmus refers to rapid involuntary eye movements presenting as one or both eyes moving from side to side, up and down or rotating or with discordance between the two eyes. Nystagmus is understood to be caused by neurological or inner ear disorders

Dizziness and balance problems constitute a major public health problem. A significant proportion of adults have had an episode of dizziness occurring with enough intensity or frequency to promote a visit to the doctor. It is one of the most difficult complaints to assess, as it is a subjective symptom of potentially numerous causes. Dizziness is often an episodic symptom, with the frequency of episodes highly variable. As a patient will very rarely have an episode in the clinic, clinicians frequently need to rely solely on the patient's (often unintentionally misleading) report of the symptom.

Accordingly, accurate diagnosis of balance symptoms is important not only to exclude potentially serious central causes but also to aid successful treatment. Unfortunately, accurate diagnosis is not possible in most cases, or delayed. Studies have shown that general practitioners (GPs) rarely failed to refer urgent cases but often failed to refer patients with persistent vestibular conditions.

Part of the difficulty is that incumbent nystagmography apparatus for detecting and recording nystagmus are large, expensive machines that are only available for use in-clinic by specialists. Due to the size, complexity and expense of these machines they are limited to laboratory or office-based use for specific tests. Those machines are not suited for use monitoring patients engaged in their everyday activities at the time of a dizzy attack.

Known ‘in-clinic’ apparatus measure eye movements using Videonystagmography (VNG) equipment. These systems are large, expensive and limited to in-clinic use. They are primarily used to record eye movements during tests that are designed to bring on symptoms.

Dizziness can be severely impairing. It can affect both physical and mental health as well as impede or otherwise affect the ability to work. Furthermore, it is significantly associated with the risk of falling and can lead to avoidance behaviour.

There are a number of underlying causes to dizziness. These include neurological, inner ear, psychiatric (especially anxiety and depression), chemical and cardiovascular pathologies. Diagnosis of the root cause is critical to exclude life-threatening conditions (such as stroke) and ensure appropriate referral for proper treatment and care for ongoing conditions.

Unfortunately, dizziness is a non-specific symptom and is often very difficult to assess. It is usually an episodic symptom with a high proportion of people reporting recurrent attacks. Episodes can last from seconds to days, and they frequently occur at home or work where there is often no means to record the symptoms of the event. As a result, the clinician often relies on the patient's account of their history of dizziness, and these accounts can be highly subjective and inconsistent.

There are four main types of dizziness: vertigo, presyncope, disequilibrium and light-headedness. Vertigo is often a defining symptom in inner ear disorders and neurological conditions but can also be present in other pathologies.

Vertigo can be objectively verified in the clinic by examining eye movements and determining the presence of a particular type of nystagmus related eye movements.

Nystagmus can be induced artificially during controlled testing in the clinic to test a limited activity range of the vestibular system

Nystagmus can occur during times of dizziness. Capturing eye/pupil movements during an episode differentiates vertigo from the other types of dizziness, and further information such as the direction and speed of the nystagmus can often lead to diagnosis that is more specific. Furthermore, the difference in movement of the nystagmus relative to each eye can further contribute useful information regarding the underlying cause.

A apparatus and process for investigating nystagmus is discussed in international patent application PCT/AU2005/001671 published as WO2006/047816. However, such an apparatus and method has at least one limitation and/or drawback.

One of the main problems with obtaining an accurate diagnosis of the cause of dizziness episodes (that can be acute) is that they can be unpredictable and frequently occur away from the medical practitioner, such as at home or work, where there is no equipment to record external symptoms of the event.

In these circumstances medical practitioners rely on the patient's account of their history of dizziness/vertigo episodes, and these accounts are highly subjective. This situation makes a complex diagnostic case even more challenging, as there are many different underlying pathologies associated with dizziness. For example, dizziness can be due to inner ear disorders, low blood pressure, neurological disorders (such as Parkinson's disease), medications, cancer, anxiety, low iron levels and low blood sugar. In these circumstances, doctors find it very difficult to identify and distinguish the underlying cause and seriousness of the dizziness and are uncertain about what type of specialist the patient needs to see.

Movement of the eyes or pupils during an episode of dizziness can give important information to the underlying pathology. The main eye and/or pupil movement observed during vertigo or dizziness is nystagmus. Nystagmus is an objective sign of the symptom of vertigo or dizziness and can help in the diagnosis of vestibular (peripheral-inner ear) or neurologic disorders. The absence of nystagmus can help the doctor focus on various other causes.

Nystagmus includes repetitive, rhythmic, to-and-fro involuntary eye movements. Such movements can include a slow drift of the eye(s) or pupil(s) in one direction (slow phase) followed by a faster correction movement (fast phase) in the opposite direction with or without a rotational component and with or without opposing nystagmus directions between the two eyes and/or pupils. Nystagmus are classified by the direction of the slow phase and can be in either horizontal (left, right), vertical (up, down) or torsional (clockwise, anti-clockwise) direction.

A single nystagmus is rarely a useful clinical sign, rather a series of similar nystagmus must be observed. For this reason, velocities of all slow waves, measured and plotted over time, can give a useful clinical picture.

Patients with nystagmus should preferably be observed for a period of 2 minutes or more to exclude the possibility of alternating nystagmus and to ensure that the current nystagmus are recorded accurately/clearly.

Several recordings during one episode can track the progress of the symptoms.

Several recordings can be used to detect recovery nystagmus (change of direction of nystagmus).

Some nystagmus episodes can last hours or even days.

Where nystagmus can be recorded and movements tracked, clinicians are provided with an objective indicator of the underlying pathology and ultimately the cause of a patient's dizziness, vertigo or imbalance; this reduces reliance on artificially induced assessment, subjective interpretation and recall of symptoms by the patient, which is often misleading and an inefficient use of time and resources

Neurologic and peripheral vestibular inner ear disorders can be differentiated based on the direction of nystagmus; purely vertical nystagmus usually indicating central origin (neurological) while horizontal nystagmus are often associated with peripheral (vestibular) conditions.

A specific example of the diagnostic utility of nystagmus is found in differentiating Meniere's disease from vestibular migraine. Research suggests that vestibular migraine is associated with nystagmus that will not change direction during an episode while those associated with Meniere's disease will change direction in the recovery phase.

It is with the aforementioned in mind that the present invention has been developed.

References to prior art in this specification are provided for illustrative purposes only and are not to be taken as an admission that such prior art is part of the common general knowledge in Australia or elsewhere.

### SUMMARY OF THE INVENTION

One or more forms of the present invention was developed with a view to improving investigation of changes in the eye(s) and/or pupil(s), such as for investigating the presence of nystagmus or other eye feature (eye and/or pupil) movement related conditions.

A apparatus according to one or more forms of the present invention can be used away from the clinic, can be wearable (e.g. temporarily for recording/sensing an episode or for a longer time period e.g. if a series of episodes occur close together), and does not need a specialist to operate it.

However, it will be understood that the apparatus may also be used more generally in oculography and for the investigation of other changes of one or more eye features e.g. in the eye, iris or pupil.

It will be appreciated that the apparatus can be used to monitor treatment outcomes. For example, once a treatment has been started (e.g. surgical, medical or physiotherapy), the apparatus can be used to monitor the affect, progress or outcome of the treatment.

The apparatus can detect/monitor if there is a change in spontaneous nystagmus or monitor the progress/recovery of a vestibular injury.

The apparatus can optionally be used to monitor any vestibular side effects after stroke and other neurologic conditions, after neurosurgical and otologic (ear) surgeries head or ear surgery (e.g. after vestibular schwannoma surgery or cochlear implantation).

It will be appreciated that the apparatus can optionally be used to monitor vertigo or dizziness symptoms during clinical trials including pharmaceutical trials.

In particular, the apparatus can optionally be used as a remote patient monitoring (RPM) apparatus that determines the presence of vertigo or dizziness symptoms in relation to the introduction of a treatment such as those that may treat dizziness and balance problems associated with the vestibular system in the inner ear, vestibular pathways or the central nervous system. Example: pharmaceuticals to treat migraine.

Furthermore, vertigo and dizziness are common side effects in many drug categories (antihypertensives, antibiotics, diuretics, NSAIDS, antidepressants, antimigrainous effects etc.).

The apparatus can be used to remotely monitor and analyse vertigo/dizziness as an adverse effect during the administration of medication and drugs.

Establishing the speed and direction of nystagmus over time is important for determining the underlying mechanisms responsible for the nystagmus. For example, up-beating and down-beating nystagmus are often associated with lesions of the brain, while left-beating and right-beating nystagmus are often associated with peripheral lesions in the inner ear. Therefore, when analysing the captured/recorded eye movements, the clinician needs to know accurately if the direction of the movement represented on the video is true.

The apparatus may unintentionally or through patient intervention be positioned incorrectly on the face, and the video recording of eye position consequently taken at a non-optimal angle. As a result, the patterns/tracings may not be truly representative of the actual direction of the eye movement.

One or more forms of the present invention includes position calibration on the user's head/face.

Calibration (or compensation) refers to referencing a particular feature of the apparatus/method to a reference or creating a reference with which to base ongoing measurements, recording or use etc. The present invention encompasses various embodiments of calibration, as described herein, such as frame positioning (windowing), apparatus positioning (correct position on user's face/head), and gaze positioning/pupil position (adjusting/compensating for angle of gaze). Other forms of calibration or compensation are envisaged to fall within the scope of the present invention.

The apparatus may include a self-contained portable face cover for at least part of a wearer's face including the eyes, such as a mask, goggles or glasses, for use in the investigation of motion of the eye(s), motion of one or more eye or pupil features and/or changes in the eye or pupil, such as presented during nystagmus.

The apparatus may include: a light omitting cover for the eyes; a suitable light source to illuminate the wearer's eyes; a digital image sensor to capture images; a data processing unit for processing the captured images; a storage medium to record the processed images; and, a communications port (wired and/or wireless) for communicating images and commands to an external receiver.

A method of using the apparatus to investigate changes of the eye is also encompassed within one or more forms of the present invention.

For example, one or more forms of the present invention may include a method of investigating changes in the eye using a apparatus that detects, measures and/or records eye movement, changes in the eye(s) or movement of features of the eye(s), the method including applying the apparatus, such as a face cover for at least part of the face including the eyes, to a wearer's face (covering the eyes), capturing images of the wearer's eyes for a period of time after said applying the apparatus; and communicating the captured images from the apparatus to an external receiver for analysis or analysing the images within the apparatus, wherein such analysis of the images is for assessing or informing diagnosis, absence or presence of a disorder or medical condition.

One or more forms of the present invention includes a method of calibrating the apparatus or set-up/position of the apparatus on a user for investigating changes in the eye(s) and/or pupil(s) of the user, such as nystagmus.

Such calibration may be conducted to determine the position of the pupil(s) of the user's eye(s) relative to the apparatus. The position needs to be known so as when the pupil(s) move, the angular degrees of movement can be accurately determined.

It will be appreciated that nystagmus are categorised by their speed and direction.

The starting point and finishing point of the pupil(s) may be used to determine direction and magnitude of movement.

Time taken to go from the eye/pupil motion starting point to the finishing point (e.g. from one to the other) can be used to determine speed and/or rate of movement e.g. as degrees per second.

Rotational motion of the eye/pupil (e.g. turning about a fixed or moving point) can be detected. Time taken for the rotational motion may be used to determine speed and/or rate of rotation and other eye kinematics.

Variation/change in angle and/or rate of change of such angle of the pupil relative to a reference axis or point can be measured. The reference axis or point can be, for example, positioned at a centre of the eye/pupil or based on a reference point/axis distant from the eye/pupil at which the eye/pupil is required to gaze.

Changes in angular velocity of eye/pupil movement are relative to each other or to a reference, rather than absolute. It is the pattern of the changes over time that is most useful.

A slow phase velocity (speed) of nystagmus is usually between 0 degrees/sec to 100 degrees/sec. A fast phase can be more than 100 degrees/sec.

One or more aspects of the present invention provides a system and/or method of setting up or calibrating the apparatus for use to help ensure correct frame position (eyes correctly positioned for viewing/recording movement of the eyes and/or correct positioning of the apparatus on the user's head/face and/or correct position within a sensor area of the image to be recorded).

Preferably, pixel count of a sensor of the camera or each said camera may be used to determine angle of gaze of the user.

Preferably, determining angle of gaze of a user need not be done in real time, but may be done so. However, such determination of angle of gaze may be determined from the recording(s) of eye/pupil position/movement and post-calculated/post-determined, and may be displayed over time, such as in a graph.

Angle of gaze may be displayed with respect to time e.g. graphing rate of change of angle of gaze.

Frame position (frame calibration or windowing): refers to the eye feature position (e.g. pupil position) within the captured video/picture frame. This preferably occurs for each eye.

Apparatus position: refers to the position of the apparatus (which may be embodied as goggles/cameras) on the user's head/face. This is in relation to the horizontal plane with respect to the user's eyes or other features of or around the eyes.

Pupil position (calibration of gaze or gaze calibration): refers to determining the correct angle of gaze (such as relative from a central position or horizontal) for the positions of the eye. This allows for the correct magnitude of eye movements to be calculated.

Frame Position (Windowing)

Frame position preferably includes detecting one or both eyes of a wearer of the apparatus is/are within a frame of reference covered by at least one image recording apparatus, such as a camera e.g. a video camera.

The apparatus may detect, sense or recognised at least one eye feature of a user's eye(s), e.g. one or both pupils. For example, the apparatus may recognise iris pattern of a user base on a previous use or recording by the apparatus for the same user.

One or more other eye feature may be recognised by the apparatus, such as pupil position, iris position, movement, patterning and/or colour or a combination of any two or more thereof.

Pupil position and/or diameter in a reference region may be identified. Zones outside of the region may be ignored or rejected by the apparatus/system/method i.e. treated as unwanted or unnecessary areas.

It will be appreciated that the eyes/pupils need not be absolutely perfectly centralized in the frame/field, rather, sufficiently centralized in order to remove extraneous areas and ensure capture of the eyes/pupils of interest is the aim.

For example, a relatively wide/large visualisation/detection field (e.g. relatively large viewing ‘frame’ within which is captured the eye as the object of interest) can be employed to ensure capturing the eye. In such a large field/frame the eye can be identified and the unnecessary frame/field outside of the eye can be removed/cut down electronically through processing or the image capture apparatus (e.g. a camera) can be zoomed/focused to the eye to remove extraneous areas.

Reducing the frame/field to cover the areas of interest (e.g. the eye) provides for higher definition imaging in the smaller field/frame area of interest. This could also be done manually e.g. by the clinician in the initial set up of the apparatus with a patient, such as by selecting a desired frame from a larger full frame picture/image.

Selecting a desired frame rate and/or frame resolution (e.g. pixel count) can be used to determine the definition and file size of the video recording.

One or more forms of the present invention utilises windowing. For example, a window derived from the image sensor.

Windowing can reduce file-size and/or increase frame rate. Windowing can, for example, utilise a window of 640×480 pixels of a sensor.

A portion of an overall sensor area may be used for windowing. That is, image data may be derived from a portion of a sensor area rather than the entire area of the sensor. Alternatively, or in addition, not every pixel across a sensor need be used.

To reduce overall size of recorded images (stills and/or video), a portion of the overall sensor may be used or selected pixels from across the overall sensor, or a combination of a portion of the sensor and some of the pixels within that portion.

Position of the eye feature(s) can be moved (such as electronically) to centralise on the sensor.

Preferably, a reduced image window (e.g. 640×480 pixels) from the image sensor is utilised to reduce file-size and increase frame rate.

One or more embodiments envisages using eye feature (e.g. pupil) location relative to the head/window/apparatus) to dynamically move the window to centralise with respect to the eyes. This will give the benefit of still having the eye in frame if the apparatus is mispositioned on the head.

Windowing can also use pupil location to dynamically move the window to centre on the eyes (e.g. during recording). This gives the benefit of still having the eye in frame if the apparatus, such as incorporated in or as goggles/glasses or other headset, are miss-positioned.

Apparatus Position

Eye feature position or eye position relative to position in the viewing frame of the apparatus (e.g. frame position relative to the eyes/eye feature) can be adjusted based on the determined position of the pupil(s), or any other feature, within the region.

The apparatus may be positionally adjusted on the face of the wearer/user or the eye/pupil position may be compensated for in the viewing frame by processing within the apparatus. For example, the apparatus may be moved on the face to position the eyes/pupils centrally with respect to viewing alignment of the camera(s).

Alternatively, or in addition, eye/pupil position can be centralized automatically by the apparatus/method e.g. electronically by moving and/or reducing visualised field/frame area to ensure eyes/pupils are sufficiently centralized.

Position of the pupils relative to one another and/or to a reference (such as at least one real or virtual horizontal reference, such as a line, with respect to the apparatus) may be used for positioning the apparatus on/relative to the user e.g. apparatus position calibration.

Height of a feature of one eye, such as a pupil, relative to a feature of the other eye, such as the other pupil, and/or to the at least one reference, may be used for positioning the apparatus on/relative to the user e.g. apparatus position calibration.

Height of eye features (e.g. pupils) relative to one another and/or to the reference can be used to determine whether the apparatus has moved or is not correctly positioned on the user, such as in a horizontal apparatus position/orientation.

If the user's eye features (e.g. pupils) are not at the same height as viewed by the camera(s), that is, the eye features (e.g. pupils) are at different relative height positions, the apparatus can be assumed to be not level.

Position of the corners of the eyes (canthi—the inner and outer canthi are, respectively, the medial and lateral ends/angles of the palpebral fissure) may be determined and used to compensate for apparatus position on the face.

One or more forms of the present invention envisages determining apparatus position/orientation, e.g. non-ideal (non-horizontal with respect to the eyes/pupils) placement of the apparatus, on a user's head.

Apparatus position can affect the calibration of the angle of gaze (e.g. pupil position), and therefore determining the position/orientation of the apparatus with respect to the user's head/face/eyes can be valuable for correcting for incorrect positioning/orientation or compensating for a particular position/orientation to ensure accuracy of any assessment/recording of eye kinematics.

By lining the centre of the pupils up—when the pupils are not aligned on the horizontal plane, one or more embodiments of the present invention encompasses either determining position over the whole video image (realign) or use the determined true horizontal plane to determine the direction of the nystagmus (e.g. in the analysis), which can be used to influence graphs and/or interpretation.

This can be achieved by using a correction factor based on the calculated rotation necessary to be aligned. Further discourse on such ‘correction factor’ is provided below.

Gaze Calibration/Pupil Position

The ellipticity, and particularly change of ellipticity, of the pupils can be assessed by using an apparatus, system and/or method of the present invention.

One or both of pupil centre and pupil shape, and/or pupil position relative to a horizontal plane/reference, may be used for calibration/position determination e.g. to set up the apparatus correctly to ensure accurate and useful image capture to aid correct assessment. Such assessment may be used for gaze calibration, apparatus position calibration/determination and/or frame position.

For example, if the pupils are not aligned on a horizontal plane, the degree of misalignment is calculated and used to either realign the pair of eye images to the true horizontal plane or as an adjustment parameter in later image post processing and analysis. Such assessment can be particularly useful where the user has mispositioned the apparatus on their face.

To accurately measure/detect the direction of the eye/pupil movements (in particular, the direction of the nystagmus) in relation to head position, it is preferable to know that when the apparatus records eye/pupil movement in a particular direction that this is the true direction of the eyes/pupils relative to the head. If the apparatus is not placed exactly horizontally aligned with the eyes, the apparatus will perceive a direction that is inaccurate, which may need to be compensated for.

Angle of gaze of the user (such as relative to horizontal or other reference) may be determined. Determination of angle of gaze of the eye(s) for a user/wearer may be based on the projected ovoid/ellipsoid shape of the pupil(s) for a particular patient/user at a given point in time.

Change in eye/pupil shape from generally circular to more oval/ellipsoid can be measured and compensated for by the image capture means and/or processing means.

Analysis of the observed pupil shape may be used to determine the confidence in the accuracy of the eye kinematics (confidence factor'). The confidence that the centre of the pupil has been accurately calculated may be represented as a real number between 0 and 1 inclusive.

This ‘confidence factor’ can be displayed alongside the eye movement graphs, in reports or any other representation of the eye kinematics.

The confidence factor may be used during analysis to modify or reject eye kinematic measurements by the clinician or the system.

For example, artefacts contributing to the ‘confidence factor’ may include, the pixel count representing the shape of the pupil image, the percentage of eyelid closure obstructing the view of the pupil, the size of the pupil, the angle of the pupil from the capture apparatus centre.

The apparatus/method may detect eye closure, for example, blinking, partial or full closure of the eyes/eyelids.

Threshold timing may be used to allow for a user blinking. For example, if eye closure is within a threshold time, the apparatus detects that blinking is occurring.

If eye closure is beyond a threshold time and/or an amount of blinking exceeds a threshold limit (count of blinks or total time spent blinking, such as a proportion of eyes open versus eyes closed), the apparatus may provide an alert and/or stop recording and/or shut down/go into sleep mode.

The apparatus being unable to sufficiently detect the eyes/pupils may be due to external light on the camera sensor(s).

Ambient/exterior light is to be omitted from reaching the wearer's/user's eyes so that the user cannot fixate their eyes on any specific feature/object. It will be appreciated that if the wearer/user does fix their gaze on an object, they can override (stop or reduce) the spontaneous eye movements to be captured.

An alert may be provided, or the apparatus may cease or not start working until the external light is removed or falls to an accepted level within the apparatus.

Known camera and/or infra-red LED positions on the apparatus may be used to determine, predict and/or estimate motion and/or location of a patient's eye(s)/pupil(s). For example, known camera and/or infra-red LED positions on the apparatus may provide known reference positions, such as distance and/or angle of separation between camera and LED(s) on the apparatus or relative to the eye(s)/pupil(s).

It will be appreciated that detection of too much light (light level or wavelength or intensity or light for a period of time, or a combination thereof) reaching a camera/sensor can be used to indicate that the apparatus is not on the face or not correctly positioned e.g. that light is leaking in.

Preferably at least one said camera may be provided below a plane of horizontal gaze of the user. For example, when a person looks down, the top eyelid often partially closes which may obscure the pupil. However, when a person looks up, their bottom eyelid does not close and obscure the pupil. It is therefore beneficial to provide at least one camera below the plane of horizontal gaze (e.g. relatively low down) and aiming up towards the eye(s).

The low camera angle image can be morphed using a keystone correction to represent a front view image.

Head movement and/or head orientation can be detected, such as by at least one accelerometer, camera image, gyroscope, magnetic sensor, or a combination of any two or more thereof, provided by the apparatus.

Such head movement and/or head orientation, and preferably eye/pupil orientation relative to the head and/or apparatus can be used to help determine a correction factor (such as mentioned above).

Head movement and/or orientation detection may be used in addition to the eye position/movement recordings. For example, when capturing eye position/movement recordings by the camera(s), head position/orientation can be synchronised with the eye position recordings, which can be used to determine what physical orientation and/or activity the user is doing at the time of recording.

Head movements may be detected (e.g. by the apparatus) and correlated as a precursor to an episode/attack or during an episode/attack.

Co-ordinating detection and/or measurement of the head and eye/pupil movements together can be used to detection of specific conditions. For example, detecting/measuring the difference between head movement and eye/pupil movement, or first detecting head movement and then detecting eye/pupil movement, can be used to detect specific conditions, such as benign paroxysmal positional vertigo (BPPV).

BPPV is generally considered to be caused by crystals forming, then aggregating and interfering within a part of the balance system in the inner ear. With BPPV vertigo is triggered by specific changes in position of the head (e.g. when turning in bed or tilting head back, looking up).

The apparatus can detect the specific head movement/s that occurred before the dizziness and then record the eye movements that ensued as a result. Not only will this help diagnose conditions such as BPPV, it can determine the ear and the specific otolith (inside the inner ear's vestibular labyrinth) that is being affected. This allows for treatment to be specifically tailored to the individual.

Glint or glints will be understood as being the reflection of light from the eye and detectable as, or indicated as, a visual feature, such as detected/seen by a camera/sensor or another person.

A glint or glints can be understood to be light reflected off the eye, such as from the pupil or iris and noticeable/detectable as a spot or patch of light in the eye. For example, a glint can be a reflection in the eye from a light source within the apparatus, such as infrared LEDs illuminating one or both eyes.

Location of at least one glint in a pupil or both eyes (e.g. in one or more pupils and/or irises of the respective eye) relative to respective pupil centre can be used to determine/estimate the centre of the eye or each eye.

One or more embodiments of the present invention utilises location of glints in a pupil or pupils relative to the respective pupil(s) centre(s) to determine/estimate the location of the centre of a patient's/user's eye/pupil.

Alternatively or in addition, eye geometry, assumptions of eye geometry, known camera and IR led locations may be used to improve accuracy and reduce the time to estimate the location, eye kinematics and the centres of a patient's pupil(s)/eye(s).

Eye geometry can be assumed and may be used to determine/estimate location of a patient's eye(s)/pupil(s).

A detection source, such as a camera, can identify the glint and its position on the eye e.g. relative to an edge of a pupil, iris or centre of a pupil, or, from the position of the glint in the eye, the centre of pupil can be determined. Consequently, for a given light source position, such as a light source fixed in position within the apparatus and illuminating the eye, the position of the glint can be used to determine eye and/or pupil centre position.

Reflection (glints) from the eyes of light from the illumination source within the apparatus may be detected by the at least one camera. Distance and/or position of the glints from the respective pupil centre may be used to determine apparatus position on the face of the user i.e. whether or not the apparatus is correctly positioned.

Distance between one or more glints and/or between at least one glint and the centre of the pupil may be used to compensate for non-ideal apparatus placement/orientation on the user's head/face with respect to eye position.

Alternatively, or in addition, relative positions of glint(s) to pupil centre may be used for gaze calibration.

For example, if the eye is further from the camera/imaging apparatus, glints in the pupil will appear closer together. Knowing the distance from the camera to the eye and assuming standard or estimated eyeball dimensions allows for the calculation of gaze angle.

One or more forms of the present invention provides the capture of information during controlled testing or simulated episodes of nystagmus e.g. in the clinic that induce nystagmus to test the current state of the vestibular equipment/system in the clinic.

One or more forms of the present invention provides an apparatus, apparatus, system or method that automatically detects eye movements, such as exhibited during nystagmus.

Statistical learning, machine learning or artificial intelligence (Al) algorithms can be employed to determine or refine the processing used to analyse captured images/image data of eye movements.

The apparatus, apparatus, system or method can be arranged and/or configured to detect patterns of nystagmus and report on them (see for example FIGS. 3A, 3B, 3C, 3D, 4A, 4B, and 4C).

Body and Environmental Metrics.

Eye information includes: any data from captured eye image data (e.g. video or still images) as current eye state (e.g. from individual frames of video or still images) or as movement or change over a period of time (eye state over time).

Eye state includes: one or more of the position, shape or size of one or both pupils; the condition/state, markings or any irregularities of the iris; irregularities in the sclera or limbal ring; any sign of infection on or in the eyeball, eyelid position, eye lid shape, any markings or infection around the eye; water around the eye; size and/or shape of the caruncle.

Eye state over time includes: changes in the position of the pupil(s) (e.g. nystagmus), changes in pupil size, pulsing movement of any parts of the eye and the surrounds thereof; eye closure (e.g. drooping eyelids or repetitive blinking); eye twitches; any changes to body parts mentioned in the eye state.

Augmented analytics includes: statistical learning, machine learning, artificial intelligence (AI) and/or any other statistical, computing or mathematical algorithms that use eye state information, eye kinematics (motion) or any other eye information associated with information from one or more sensors, including environmental sensors (e.g. temperature, pressure, atmospheric pressure, humidity, air/wind speed, magnetic field, direction, orientation (e.g. up, down, prone)—such as relative to a ground plane or horizontal/vertical, body movement sensors (such as head movement from accelerometers), and biomedical sensors (such as blood pressure, body temperature)).

Analytics, e.g. augmented analytics, can incorporate sensor information obtained/derived from the apparatus and/or obtained/derived from one or more sensors external to the apparatus.

Sensor information and/or analytics may be used to determine:
Eye state in relation to environmental, body and/or head movement, and/or body metrics, or any combination thereof.
Eye state over time, such as derived eye kinematics, in relation to the environment, body and/or head movement, and/or body metrics.
Threshold in which changes to any aspect or aspects of the environment, body, head, body movement, head movement and/or body metrics, are associated with or cause changes in eye state over time. For example, a threshold at which reduction or increase in atmospheric pressure induces nystagmus.
A combination of thresholds of two or more changes in body state, environmental state and/or body metrics relates to or causes changes in eye state or eye state over time.
Detection of eye behaviours related or that may be symptomatic of at least one medical condition (e.g. an underlying medical condition exacerbated by a change in environmental or body metrics or a medical condition caused by such a change).

One or more embodiments of the present invention incorporates monitoring of the environment and/or sensing/providing environmental information, such as to be used in combination with data/information directly from or derived from the apparatus.

Environmental information/data can include underwater, high altitude, space flight, outer space and space living conditions(e.g. reduced/low gravity living; artificial, centrifuge and/or rotation induced gravity) obtained/derived information/data. Such information/data can include ambient temperature, temperature change, air/water pressure and/or humidity or water density/salinity (e.g. for underwater).

Sensors for sensing environmental conditions can include detection of gravity, changes in gravity, direction and/or amount of gravity, radiation (e.g. type, level and/or change), atmospheric pressure, hydrostatic pressure, temperature, humidity, pollen count, oxygen level, presence of one or more gases, and/or the level of any gas or combination of gases.

Environmental and/or body metrics may be combined with measurement/detection of eye state and/or eye kinematics, such as over time, to assess the influence of environmental factors on the neurological or vestibular system. For example, the effects on the neuro-vestibular system of underwater pressure or the effects of reduced/no gravity or changes in pressure/gravity over time.

For example, for assessing the effects on a person of high altitude and/or space flight — eye position/changes in eye position (such as nystagmus) in relation to head position with the change in environmental conditions (reduced/low/changes of gravity and/or air pressure and/or temperature)

Body metrics can include monitoring/measuring body functions and activities. Such body metrics can include one or more of measuring blood pressure, body temperature, pulse, ECG, EEG, blood glucose level, hearing level/ability, lung capacity, exercise sensors and information from body scans/images (MRI, PET, CT, X-ray, photogrammetric).

Body metrics can be sensed, measured and/or used in combination with eye kinematics/eye state measured by or derived from the apparatus (e.g. from the apparatus—pre or post processing) to determine/assess changes in the body that occur before and/or during neuro-vestibular dysfunction or changes in neuro-vestibluar function.

The combination of body metric information/data with eye state data and/or eye kinematics can be used to determine one or more triggers of vestibular system dysfunction, including long term and/or short term affect vestibular injuries/changes have on the body. This can help to understand how the vestibular system is affected by changes in the body and how the body is affected by the vestibular system.

Augmented analytics in combination with body metrics, identified patterns and triggers can help to determine the underlying cause of neurological and/or vestibular dysfunction. For example, to determine that hyperventilation precluded nystagmus eye movements.

Eye closure information/data can indicate fatigue and the presence of nystagmus indicates balance dysfunction. Together or separately this information can indicate whether a body meets fitness thresholds or to perform one or more particular functions. For example, the combined information can be used to assess a patient for fall risk after surgery or the ability to safely perform certain tasks after or during an extended period in space, undergoing space flight, a change in gravity on the body, or living in a low/reduced gravity environment e.g. compared to mean or sea level gravity on earth.

Sensors on the body (torso, one or more of limbs, hands, feet, etc.) and head can include one or more head movement sensors (such as on the apparatus or external to the apparatus of the present invention), internal of the body (implanted, inserted or ingested), external of the body (mounted to the body or worn). Sensors may also be external and remote from the body, such as image capture means for photogrammetry.

Sensors can include accelerometers, gyroscopes and electro-static accelerometers.

The vestibular system can be triggered by changes to head position (roll, pitch, yaw) and in the normal case results in eye movements opposite the direction of movement. This is called the vestibular ocular reflex and it is a gaze stabilising reflex. If the vestibular system is compromised (e.g. during a change in gravity) then the vestibular ocular reflex acts accordingly in an expected manor. Measuring both the head movement and corresponding eye movement can be used to assess whether the vestibular system is functioning correctly. Any inconsistency with the response to head movement indicates a malfunctioning vestibular system or a vestibular system working differently/unexpectedly.

Body metrics and body/head movement can be used in combination with environmental measurements/information/data to determine/assess the effect on the neuro-vestibular system of the environmental changes/conditions.

Likewise, detected changes in the state or function of the neuro-vestibular system can be used to assess why a change in the environment/body metric and movement occurred. For example, analysing the vestibular ocular reflex (which considers eye and head movements) in relation to changes in gravity or radiation on the body. Such assessments could be used to determine a gravity threshold where the vestibular ocular reflex becomes affected.

Security/Identification

One or more forms of the present invention may obtain eye status information for biometric security/identification purposes.

One or more images obtained by an embodiment of the present invention can be used to identify the wear or features of the wearer for identification e.g. for authentication, security, access control, authority to control/use equipment, authority to allow others such access/use etc.

Portability of a user wearer apparatus according to an embodiment of the present invention allows the apparatus to be deployed readily without the need for a fixed installation.

For example, iris imaging within the apparatus can exclude extraneous light and desired illumination can be provided to obtain desired image quality.

Sclera imaging, limbal ring imaging, eye shape imaging, distance between eyes/pupils, position, size and complexity of blood vessels in the eye, may be used to assess the person for security/identification purposes.

Health Status

Detection/measurement of eye state or changes in eye state by the apparatus/system of the present invention can be used to assess general health and any condition affecting the subject person, such as drug use, alcohol use, stress, fatigue or illness.

Such information/data can be combined with information from other sensors, such as external or internal body sensors.

Sclera imaging, eye shape imaging, distance between eyes/pupils, position, size and complexity of blood vessels in the eye, and any changes thereto, may be used to assess the person for health/change in health purposes.

One or more embodiments may include assessing data relating to head movement and eye or pupil motion, which may include assessing data relating to at least one measured body function and/or at least one environmental factor. The at least one body function may include at least one of heart rate, blood pressure, body temperature. At least one environmental factor may include at least one of ambient temperature, air pressure, humidity, location.

One or more embodiments may include determining at least one threshold, safe range, cut-off. The at least one threshold, safe-range or cut-off may include determining one or more of a safe threshold for a person conducting an activity, a threshold for time spent in or presence in a particular gravity or change of gravity.

A further aspect of the present invention provides a system for use in assessing changes in eye state, the system including a wearable apparatus for covering a wearer's eyes that detects, measures and/or records eye movement, changes in the eye(s) or movement of features of the eye(s), the system including image data capture means for capturing image data of motion or at least one feature of the wearer's eyes for a period of time, communication means for communicating the captured image data, and processing means for use in assessment or informing diagnosis, absence or presence of a disorder or medical condition.

The apparatus may be configured to capture a starting point and finishing point of the pupil(s) to determine direction and magnitude of eye or pupil movement. The system may be configured to capture image data and determine time taken for the pupil(s) to go from the starting point to the finishing point for the system to determine speed and/or rate of movement.

The system or apparatus may include use of a reference axis or point determined at a centre of the respective eye(s)/pupil(s) or based on a reference point/axis distant from the eye(s)/pupil(s) at which the eye(s)/pupil(s) is/are required to gaze.

Pixel count of a sensor of a camera or each said camera of the apparatus can be used to determine angle of gaze of the wearer.

Processing means can be provided to determine angle of gaze of the eye(s) from image data and/or image data recordings of eye/pupil position/movement.

A further feature includes a reference region applicable to pupil position and/or pupil diameter. At least one zone may be outside of the reference region, wherein such at least one zone is ignored or rejected or treated as unwanted or unnecessary during processing.

A previsualisation/detection field may be within which the eye or pupil is captured as an object of interest for capturing the eye in image data and unnecessary frame/field beyond the eye or pupil is removed/reduced.

Electronic processing or zoom/focus of the image capture apparatus can be provided to remove extraneous areas.

A portion or window of an overall sensor area of the image capture apparatus may be provided for windowing such that captured image data is derived from a portion of a sensor area. Preferably, the portion or window is central with respect to the eye(s)/pupil(s) to frame the eye(s)/pupil(s) in frame.

One or more of pupil centre, pupil shape or pupil position may be relative to a horizontal plane/reference for calibration/position determination.

Measurement means, such as one or more sensors and/or algorithms, can be provided for measuring/imaging variation in eye/pupil shape from generally circular to ovoid/ellipsoid, and compensation means for compensating for non-circular irregularity via the image capture means and/or processing means.

One or more of pixel counts or patterns can represent the shape of the pupil image, percentage of eyelid closure obstructing the view of the pupil, size of the pupil or angle of the pupil from the capture apparatus centre as a confidence factor for determining accuracy of eye/pupil kinematics.

A light detector can be configured to detect ingress of external light and instigate an alert, or the apparatus is stopped operating or is prevented from operating until the external light is removed or falls to an accepted level within the apparatus.

The apparatus can include or be a wearable apparatus, such as goggles, face mask, headset etc.

At least one image capture apparatus may be provided below a plane of straight-ahead/horizontal gaze of the user/wearer.

At least one measurement means may be provided to measure one or more of environmental metrics, body metrics or head metrics, in addition to information/data obtained or derived from image capture information/data from the apparatus, to assess neurological and/or vestibular condition or activity.

A further aspect of the present invention provides calibration (or compensation) of a wearable apparatus for use in assessing a neurological or vestibular condition, including referencing a particular feature of the apparatus to a reference or creating a reference with which to base ongoing measurements, recording or use. One or more embodiments of calibration includes frame positioning (windowing), apparatus positioning (correct position on user's face/head), and gaze positioning/pupil position (adjusting/compensating for angle of gaze). Other forms of calibration or compensation are envisaged to fall within the scope of the present invention. Calibration (or compensation) may include one or more of:
Eye feature position (e.g. pupil position) within the captured video/picture frame. This preferably occurs for each eye.
Apparatus position calibration: position of the apparatus (which may be embodied as goggles/cameras) on the user's head/face. This is in relation to the horizontal plane with respect to the user's eyes or other features of or around the eyes.
Pupil position (calibration of gaze or gaze calibration): determining the correct angle of gaze (such as relative from a central position or horizontal) for the positions of the eye. This allows for the correct magnitude of eye movements to be calculated.

Assessing effects of space travel/time spent off-earth (e.g. reduced gravity environments relative to earth gravity)

One or more embodiments of the present invention finds application in assessing space travellers prior to, during and/or after space travel or time spent on non-earth planet, extra-terrestrial body or an off-earth installation, vehicle or habit.

Embodiments of the present invention may be employed to assess long term effects of space travel on space travellers returned to earth, or the effects of time spent in space in reduced gravity environments (such as space craft, space stations, non-earth planet habitats e.g. the Moon or Mars).

### BRIEF DESCRIPTION OF THE DRAWINGS

One or more embodiments of the present invention will hereinafter be described with reference to the accompanying Figures, in which:

FIGS. 1A to 1C show examples of horizontal, vertical and torsional eye movement with respect to nystagmus.

FIG. 2 shows a chart of nystagmus related to horizontal eye movement (speed and direction) plotted over time on a graph with horizontal eye position on the y axis and time on the x axis.

FIGS. 3A to 3D show patterns (e.g. speed and direction of nystagmus over time) of captured nystagmus utilising a apparatus/method according to at least one embodiment of the present invention.

FIGS. 4A to 4C show an alternative arrangement for displaying captured nystagmus patterns (speed and direction over a time period) using radial stacked histogram charts according to at least one embodiment of the present invention.

FIGS. 5A to 5D relate to steps taken for adjusting for misalignment of the apparatus relative to eye position of a patient according to at least one embodiment of the present invention.

FIG. 6 shows a side sectional view of a apparatus according to an embodiment of the present invention.

FIGS. 7A and 7B show examples of glints reflected in the eye of a wearer as captured by image capture means of a apparatus according to an embodiment of the present invention.

FIGS. 8A and 8B show an eyeball with its pupil in two different locations. Shown are 2 gaze vectors, the lines from the centre of the eye through the centre of the pupils.

FIGS. 9A, 9B, 9C and 9D show an eyeball with its pupil being 5 mm diameter in two locations being centred with respect to the capture apparatus image and being 45 degrees upwards from the capture apparatus image.

FIG. 10 shows an alternative embodiment of the present invention.

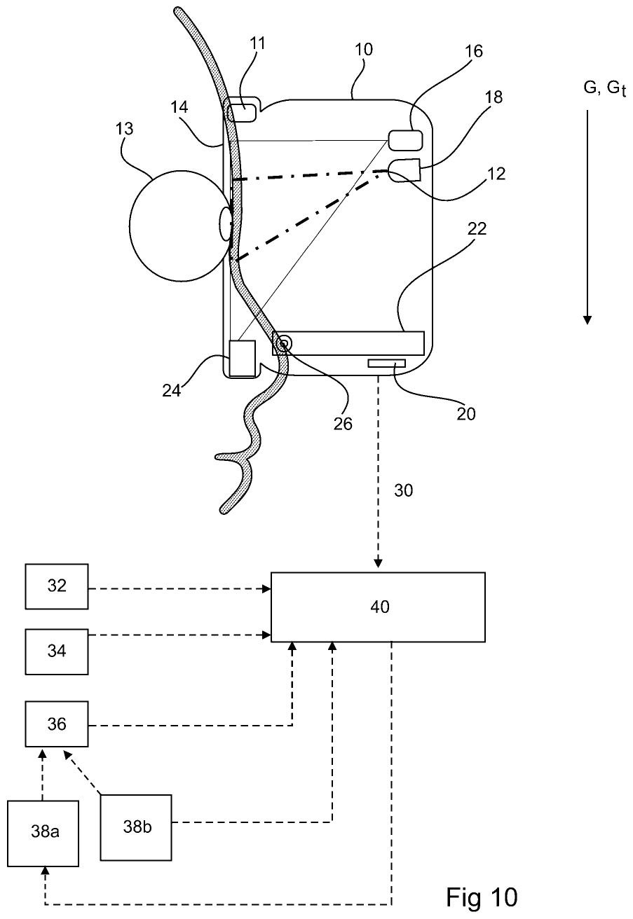

FIG. 11 shows an example of an embodiment of the present invention incorporating environmental sensing/measurements.

FIG. 12 shows an example according to an embodiment of the present invention incorporating body sensing/measurement.

It will be appreciated that one or more embodiments of the present invention can incorporate environmental sensing/measurements and body sensing/measurements.

FIG. 13 shows an example of capturing head movement and eye motion data according to an embodiment of the present invention.

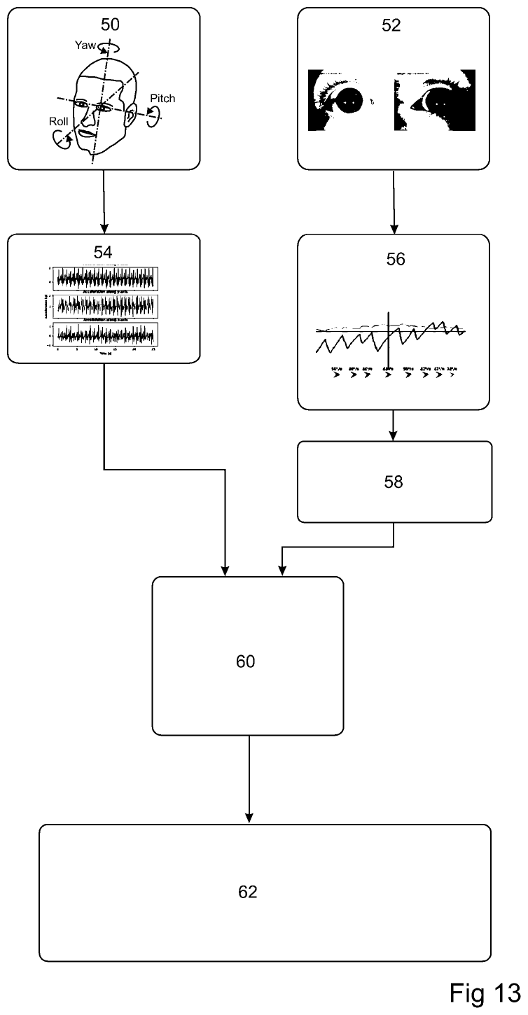

FIG. 14 shows an example of a patient experience storyboard' flowchart and employment of one or more embodiments of the present invention.

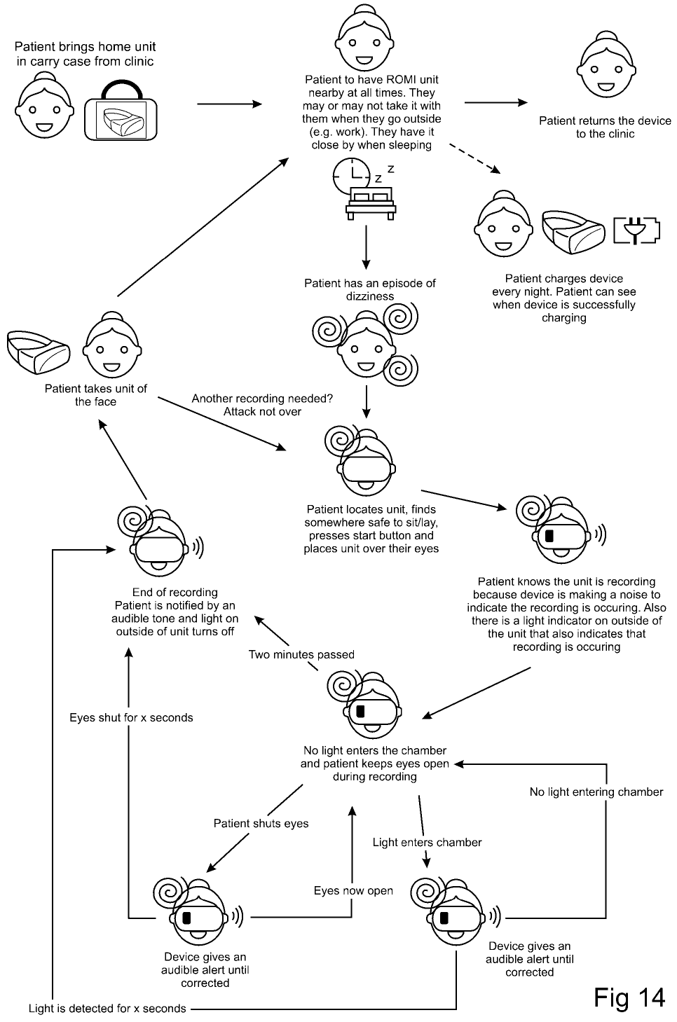

### DESCRIPTION OF PREFERRED EMBODIMENT(S)

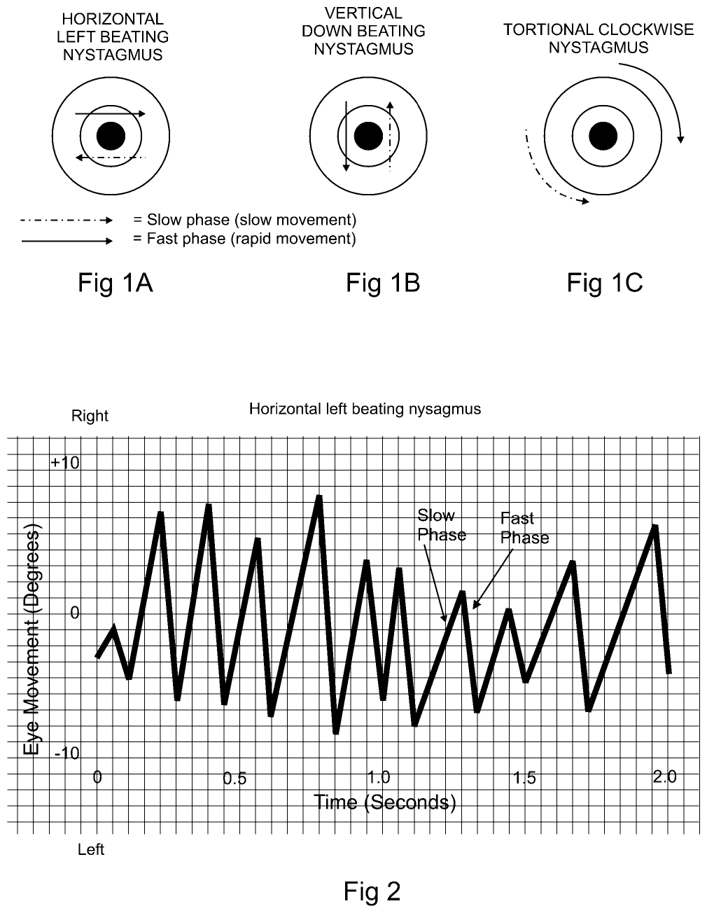

As shown by way of example in FIGS. 1A to 1C, typical examples of nystagmus.

FIG. 1A shows nystagmus presenting as horizontal nystagmus e.g. left nystagmus where the eye moves slowly to the patient's right and rapidly returns horizontally to the left.

FIG. 1B shows nystagmus presenting as vertical nystagmus e.g. down beating nystagmus where the vertical slow eye movement upward is followed by rapid vertical downward rotation.

FIG. 1C shows torsional nystagmus e.g. clockwise nystagmus with the eye slowly rotating anticlockwise followed by a rapid counter torsional clockwise rotation.

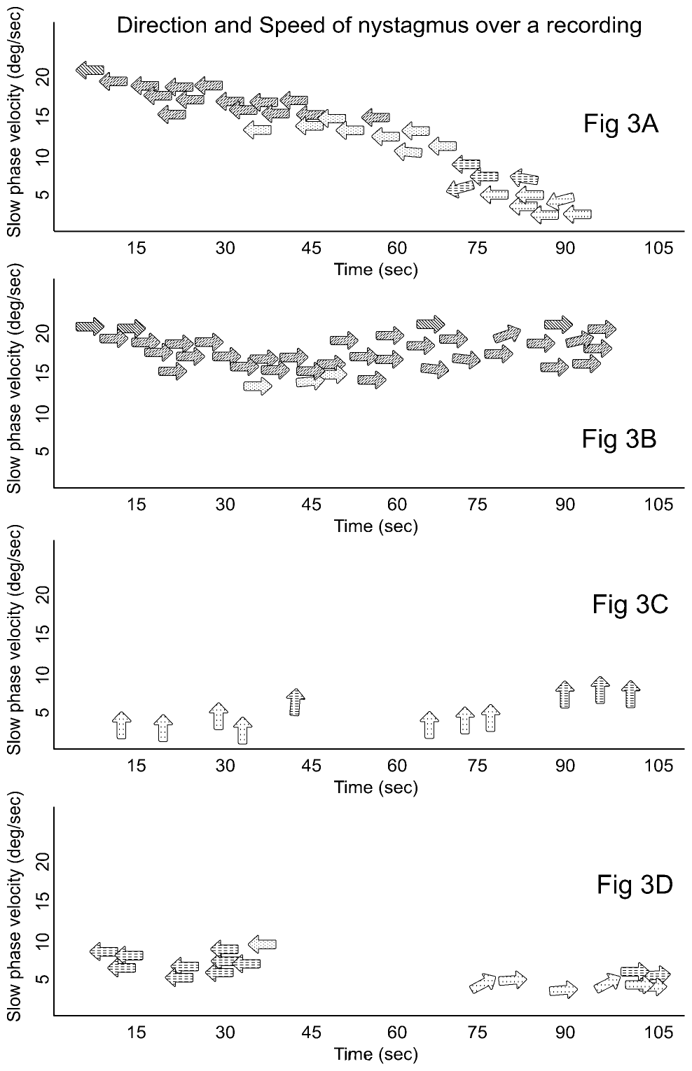

FIG. 2 shows a chart of nystagmus related eye movement (e.g. speed and direction of movement) plotted over time on a graph with horizontal eye position on the y-axis (gaze right at the top and gaze left at the bottom) and time on the x-axis. This figure shows left beating nystagmus.

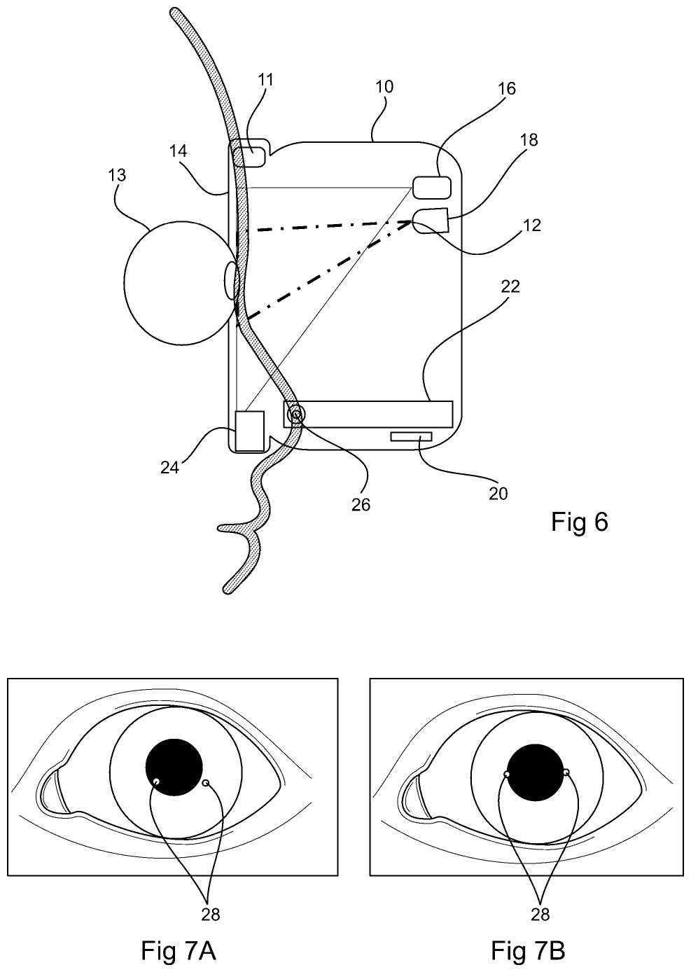

Nystagmus over time can be plotted a number of ways. Horizontal and vertical tracings are currently adopted. There are also, new ways to display the data. This is driven by knowing exactly the pupil position (due to realignment of the horizontal plane from lining up the pupil or using glints (28 FIGS. 7A, 7B)) and therefore accurately know the direction and speed of the nystagmus.

The features of nystagmus such as shape, amplitude and frequency can be analysed. Nystagmus are usually described by speed of eye movement (e.g. of the slow phase) and direction of eye movement (e.g. of the fast phase). For example, 16°/sec right beating nystagmus. Instead of direction they can also be defined in relation to gravity (geotropic: towards earth, ageotropic: away from earth).

According to one or more embodiments of the invention, captured nystagmus motions can be charted/displayed showing direction and velocity of movement over time.

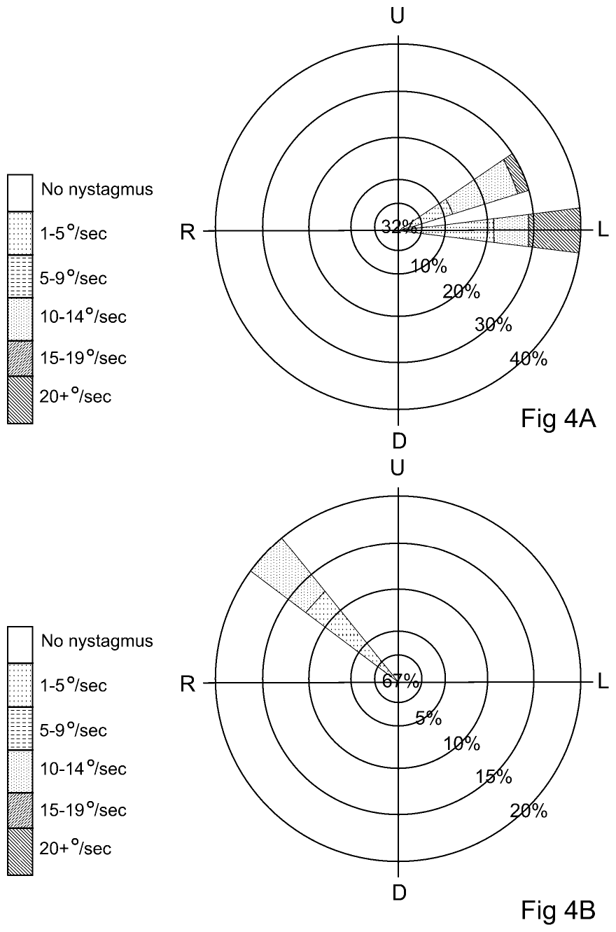

For example, FIG. 3A shows by way of example charted strong right-beating nystagmus that reduces in velocity over a minute and a half of time.

FIG. 3B shows an example of charted captured strong persistent left-beating nystagmus.

FIG. 3C shows charted captured mild persistent up-beating nystagmus.

FIG. 3D shows charted captured direction changing nystagmus with respect to time. Moderate right-beating nystagmus for 40 seconds followed by a direction change to left beating nystagmus.

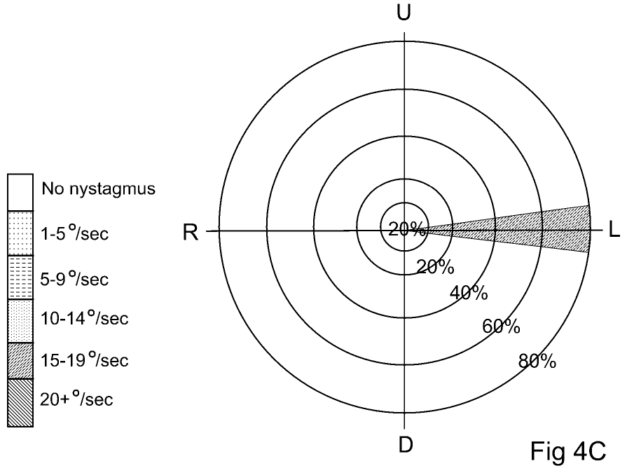

FIGS. 4A to 4C show alternative visualisation of nystagmus results as captured by the apparatus/method of the present invention.

In particular, FIG. 4A shows left beating nystagmus (some with slight upward component) of varying velocity.

FIG. 4B shows occasional upward and to the right beating nystagmus of mild to moderate velocity.

FIG. 4C shows strong left beating nystagmus.

Being able to visualise the pattern (speed and direction) of nystagmus during an attack significantly aids in diagnosing the underlying cause of the attack. The present invention provides a new modality of capturing, measuring and displaying time of attack nystagmus and being able to view and present the pattern (speed and direction) significantly improves the assessment methodology.

It is important to have the apparatus correctly positioned on the patient's head/face or to be able to determine position of the apparatus on the patient's face and compensate for sub-optimal positioning to ensure useable/correct results.

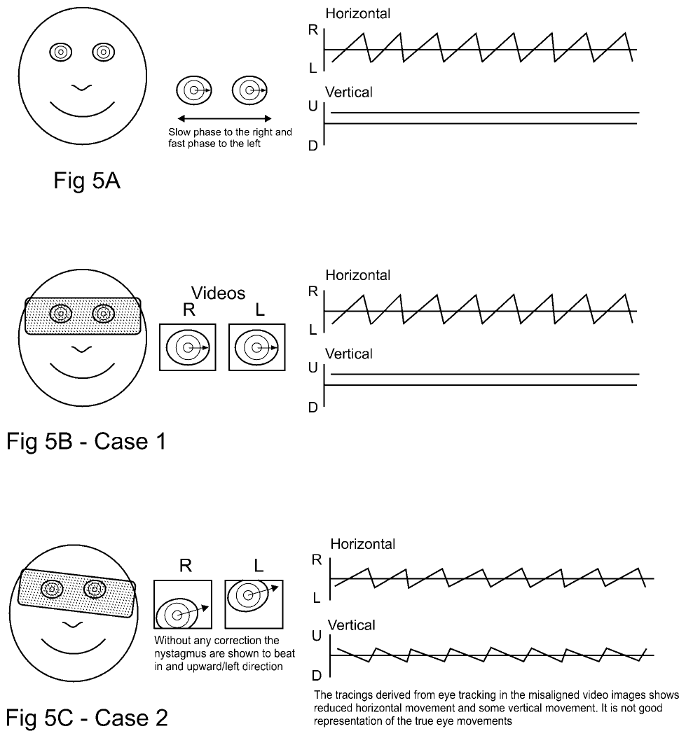

FIGS. 5A to 5D show a technique according to one or more embodiments of the present invention that can be employed to correct misalignment in recordings due to incorrect apparatus placement.

In this example, the patient has pure left beating nystagmus, as shown in FIG. 5A. Eyes move slowly to the patient's right and then a fast movement back to the patient's left. In this embodiment, the tracing should show left beating nystagmus with no movement on the vertical scale. The upper tracing shows the horizontal (left and right) movement and the lower tracing represents the vertical (up and down) movement of the eyes.

FIG. 5B shows an example (Case 1) of what happens to the eye movements in FIG. 5A when the apparatus is horizontally and vertically accurately aligned with eyes. In this case, the eye movement recordings show the true direction of eye movement (e.g. right and left movement are true right and left movement). This translates to accurate tracings. Note that there is no vertical component shown in the tracings as there was no vertical eye movement.

FIG. 5C shows an example (Case 2) of what happens to the eye movements in FIG. 5A when the apparatus is incorrectly positioned. That is, the apparatus is not horizontally and vertically aligned with the patient's eyes. In this case the tracings do not reflect the true direction of the nystagmus. Note that there is a vertical component shown in the tracings although no vertical eye movement occurred.

When the apparatus is not positioned correctly (Case 2), the misalignment (correction factor) can be measured and used to recalculate eye position so the tracings reflect the true direction of the eye movements. This is explained in FIG. 5D.

FIG. 5D shows that using the captured images (video images or series of still images), the degree of misalignment is calculated. This can be achieved by using the pupil centres as an indication of the horizontal plane

Other methods within the present invention can also be used to determine the horizontal plane. These include determining the position of the corners of the eye or any other feature of the eye and surrounds. For this example the pupil centres are used, as shown in FIG. 5D.

As shown in Steps 1 to 4 of FIG. 5D:

In step 1, the degree of misalignment of the pupils from horizontal is determined.

In step 2, correction is applied by rotating the image/video back by the degree of misalignment so that the pupils are horizontal and the frame is non-horizontal.

In step 3, frame correction is applied to centre the pupils within the frame.

In step 4, the corrected tracing of eye movement during nystagmus are captured.

As an alternative in steps 2 to 4, the video can be kept the same and the calculated degree of misalignment can be used to recalculate the tracings.

It will therefore be appreciated that the present invention envisages many ways to plot the nystagmus. For example, horizontal and vertical tracings can be used.

Embodiments of the present invention also envisage measuring, plotting and/or assessing nystagmus relative to gravity. Eye motion (optionally also head movement) can be tracked/measured and compared with gravity (gravity direction, strength of gravity, change in strength and/or direction).

The nystagmus of lateral canal BPPV can be categorized as either always towards the ground (geotropic) or always towards the sky (ageotropic).

Alternatively, knowing the position (due to realignment of the horizontal plane from lining up the pupil as well as the calibration data using glints 28 (FIGS. 7A, 7B) or ovoid shape of pupil as example), the direction of the nystagmus can be plotted.

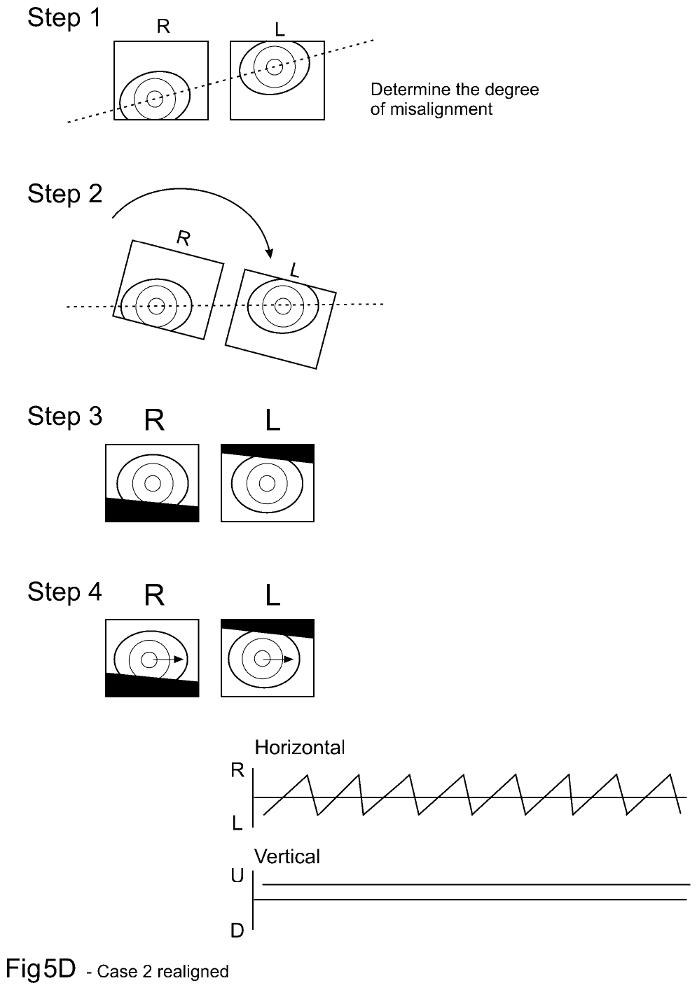

FIG. 6 shows a side sectional view of a apparatus 10 according to an embodiment of the present invention.

In particular, the apparatus 10 is provided as a wearable apparatus, such as goggles or other type of headset.

When the patient is experiencing an attack of vertigo, dizziness or imbalance they place the apparatus in front of their eyes 13. The apparatus is turned on, such as by an on-off switch 11 provided on the apparatus (or remotely by wireless communication control).

Two lens(es)/cameras 12 record the eyes/eye features (e.g. movement of the pupils).

There is preferably a cover to the lenses for each lens. The cover may be opaque to the wearer/user so that the patient's eyes are in complete darkness with no internal reflections in order to avoid any visual distractions. Recording can of course still be made through the opaque cover i.e. the cover is opaque the user and not to the lenses/cameras.

A seal 14, (such as a flexible, foam, plasticized or rubberized seal) is preferably provided around a face side periphery of the apparatus to prevent external light entering the cavity so the patient cannot see any visual distractions.

A separate, removable adapter (such as a face mask) can be removably attached to the apparatus in order to accommodate different head sizes/face shapes. The removable adapter (e.g. face mask) can be disposable or cleanable. The removable adapter (e.g. face mask) may include a surround for contact with the user's face, the surround may be removable from the apparatus.

Preferably the surround and/or adapter (e.g. face mask) is formed of or includes a flexible material or combination of materials, such as a polymer, e.g. silicon, rubber and/or foam based. The face mask may be shaped so as to fit around the user's eyes and over their nose e.g. over the bridge of the nose, similar to googles for use in water or skiing, for a comfortable light omitting fit e.g. sealing around the contact area with the skin. The adapter (e.g. face mask) can be of a light blocking material to prevent light passing to the eyes and image capture means that would consequently impair image capture of the eyes.

The patient's eyes are illuminated by a light source 16, such as by one or more infrared LEDs, incorporated as part of/attached to/within the apparatus. This is so the wearer/user cannot fixate on any point, therefore overriding the spontaneous eye movements.

One or more cameras 18, such as video cameras, (e.g. one for each eye, or a single camera and a mirror arrangement) record the patient's eye movements (nystagmus) during an attack episode e.g. a vertigo attack.

The video recordings are stored, such as on the memory apparatus 20, which may preferably be located mounted within or on the apparatus.

Preferably the memory apparatus is connected for data transfer with electronic circuitry 22 controlling the apparatus.

When the patient's episode attack has finished, the apparatus is removed from their head.

The video recordings can be provided to a processing system.

The recordings are preferably compressed and eye movements logged.

Processing of the eye movements can occur within/on-board the apparatus.

Other data, such as the quality of the image, how long the pupil was open, can be stored and can be analysed on the unit or transmitted to be analysed remote from the apparatus, such as at a clinic.

Capture/recording of eye position/movement (optionally with head position/orientation measurements) can be transmitted to a remote location, such as the ‘cloud’ or remote server, to be processed there.

The apparatus can be battery powered, such as by an on-board battery 24 and/or can receive power through a power connection 26.

Eye kinematic measurements, such as gaze angle and rate of movement/change of movement/rate of change of movement, can be calculated and used to assess presence of a medical condition, such as nystagmus or other neurological disorder.

Angular velocity can be determined by calculating rate of change of gaze angle (e.g. change of angle between gaze vectors for a given time interval).

Further, angular acceleration can be determined by calculating the rate of change of angular velocity for a given time interval.

Angular jerk can be determined by calculating the rate of change of angular acceleration for a given time interval.

Angular jerk measurements can be used to detect when the eye movement transitions from a slow phase to a fast phase and or when the eye movement changes direction.

Definition: ‘within phase’ means sub-periods or intervals of time within a traditionally identified phase of a nystagmus e.g. slow phase or fast phase, such as shown by way of example in FIGS. 2, 5A and 5B.

Analysis of the eye kinetics within phases (e.g. slow phase) may find eye movement patterns relating to the speed, position, direction, changes of direction, acceleration of the pupil (or other eye features) and other parameters.

For example, the slow phase of a horizontal right beating nystagmus, may have smaller periods of vertical up beating nystagmus or some other notable feature that are contained within the time interval defining the slow phase.

Eye kinematics including the angular jerk measurements can be used to identify ‘within phase’ transitions that may not be detected with incumbent clinic eye tracking apparatus. These ‘within phase’ transitions will lead to new research opportunities for identifying and diagnosing diseases with dizziness symptoms.

Identifying ‘within phase’ eye kinematics may lead to better diagnosis of complex cases. This includes cases where multiple diseases, conditions or factors may contribute to or confound dizziness symptoms when using traditional nystagmus analysis.

Furthermore ‘within phase’ eye kinetics may lead to further information about the underlying pathology in patients with vestibular dysfunction. For example it may give information as to the functioning of the vestibulo-cerebellar pathway and/or the vestibular nuclei.

The present invention includes algorithms to detect traditional nystagmus, their related eye kinematics and ‘within phase’ eye kinetics.

The apparatus and software patent includes algorithms to detect ‘within phase’ eye kinematics.

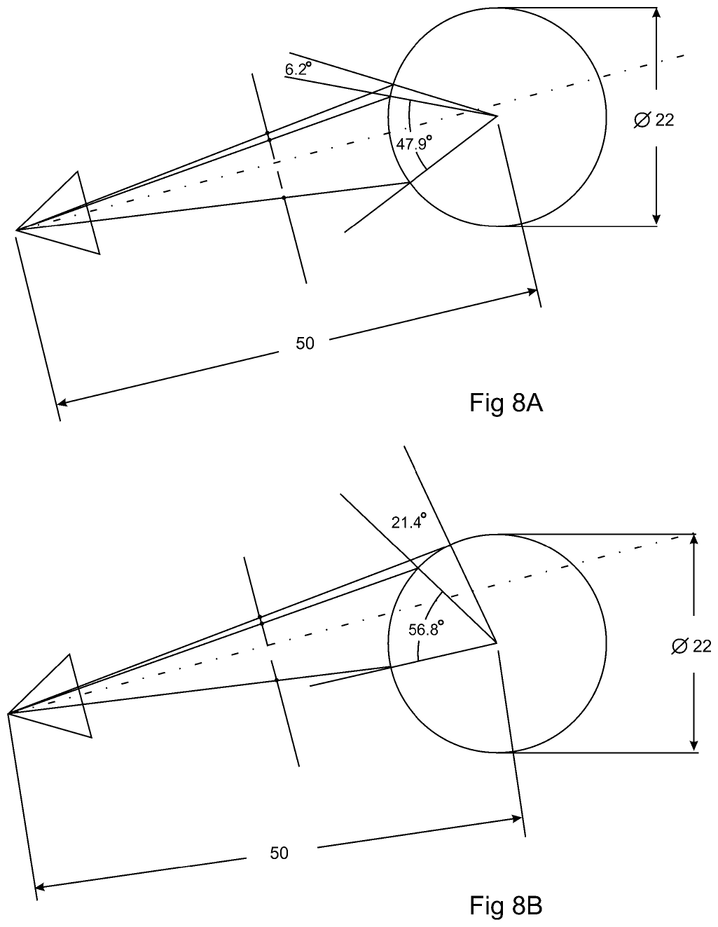

FIGS. 8A and 8B show identical pupil locations relative to the image capture means (e.g. camera). They illustrate how differences in the calculated eyeball centre result in errors in the angles between gaze vectors and therefore errors in angular velocity.

Results/statistics (such as summary or full statistics) of one or more patient episodes of nystagmus based on the eye movements can be provided by the apparatus or as processed remotely and displayed on a local display (local to the apparatus) or transmitted to a remote display (e.g. tablet, pc, web browser client) . For example, the apparatus/method may categorise the extent or type of nystagmus (e.g. 6 degree per second right beating) and display the type or types (or pattern) to give a clinician instant information/knowledge. The apparatus may include or have access to processing means to process and supply a suggested test.

The apparatus 10 for such remote oculography monitoring and software together provide medical specialists with a unique custom solution to aid their diagnosis of vertigo, dizziness and balance symptoms as a result of neurological and vestibular (inner ear) conditions.

One or more embodiments of the present invention provides a portable apparatus designed to obtain and/or record patient's eye movements during episodes/attacks of dizziness away from the clinic. That is, patients can administer the apparatus themselves when they are at home, work or during recreational activities.

The medical specialist can review the captured data. This application allows specialists to view and analyse the uploaded patient data as well as collaborate with colleagues regarding the assessment of patients' symptoms.

The system allows for the day-to-day administration and collection of patient information. Stored information includes one or more of:
eye movement video sequences;
3D orientation and position of the head during the episodes;
eye kinematics and tracking data;
Other eye feature information (e.g. pupil size);
history;
questionnaire data; and
information pertinent to diagnosis.

A software platform facilitates use of the apparatus and can help to identify patients for specific treatment options and enables research from the captured data including severity, timing and location of episodes.

Additionally, the system can include monitoring the use of a number of the apparatus, and can oversee the setup and administration of clinics employing the apparatus.

The apparatus 10 can include goggles configured to monitor and/or capture patient's eye/pupil movement(s), such as during episodes of dizziness. When the patient begins to experience an episode, the patient wears the apparatus. The apparatus commences recording the episode automatically or when switched on.

It is appreciated that users/patients will have the apparatus for 2-4 weeks and clinics will possess a number of the apparatus to deploy to a respective number of users/patients, with potential for spare apparatus being kept on hand.

Alternatively, the patient or each patient may keep the respective apparatus. The patient could choose to keep the apparatus permanently (e.g. for a cost) and only the specialist/clinician can provide a report/analysis when the apparatus transmits new/updated measurements/data of one or more nystagmus episodes.

In use, the apparatus captures video of the eye kinematics and preferably patient position (e.g. accelerometer, gyroscope) data.

Preferably, the apparatus may perform on-board processing that occurs during or after the video capture. For example, the on-board processing may detect if the patient closes their eyes during the recording and/or after the capture may compress the video data and/or log particular events, such as variability in eye movement or eye closure or periods of non-movement between bouts of eye movement.

The captured data can be sent (streamed, sent in packets or sent as a whole) from the apparatus to a platform e.g. via hard wire or wireless, such as via a mobile phone network.

If a mobile network is not available, the apparatus can include the facility to store the data on-board to be downloaded e.g. at a later time (such as via USB, internal or removable storage).

Preferably, software on the platform is cloud based and accessed through a web-app.

By way of explanatory example, a patient can have custody of the apparatus permanently or for a time period. When the patient senses or begins experiencing an episode of dizziness, the patient puts on the apparatus and commences recording eye movements using the on-board image capture means.

The patient can receive a visual, audible and/or vibration alert that the apparatus is recording. If the patient shuts their eyes during recording or if ambient light enters the apparatus thereby affecting recording, the apparatus gives an alert to the patient.

If the eyes are closed and/or light entering continues for a period e.g. 10 seconds, the recording can be halted/paused and the patient notified. Alternatively, if the apparatus successfully records eye movement images for a predetermined threshold period, say, 120 seconds, the apparatus could indicate that successful recording is completed. The patient can cease recording. The captured image data is stored, processed on-board and/or sent for remote processing.

With respect to the clinic side for deployment of the apparatus, a patient is instructed when and how to use the apparatus. After sufficient episode recordings are completed, the patient returns the apparatus to the clinician or, as otherwise arranged, keeps the apparatus.

Recorded data may be obtained directly from the apparatus (as processed data, semi-processed data for additional processing, or raw data for subsequent processing) and the obtained data is analysed. The image data can be transmitted from the apparatus for remote processing, such as by hard-wired, local wireless transmission e.g. Wi-Fi and/or mobile phone network link for remote processing and/or storage and/or analysis.

Episode recording may be monitored in real or near real time by clinicians, such as by automatic detection and transmission of recorded or direct image data as an episode occurs.

The present invention provides advantages of:
Reduced contact time with patients and increase the likelihood of arriving at a definitive diagnosis.
Ability to diagnose more patients due to (a) the reduced occurrence of repeat appointments with existing undiagnosed patients, and (b) shorter appointments with all patients.
Provide a low cost assessment option requiring minimal capital expenditure that provides a rapid return on investment.
Improved patient care.
Provides a source of income for the clinics.

For the patient, the value proposition for apparatus/system is:
The apparatus/system provides a more convenient method of recording their spontaneous nystagmus.
Shorter waiting times for diagnosis and treatment.
A quicker path to the correct specialist and an increased likelihood of a correct diagnosis.
Empowerment of the patient by being actively involved in identifying the cause.
Symptoms are recorded objectively and not reliant on patient report, therefore providing credibility to the patient's medical complaint.
Lower costs due to the need for fewer tests.
Fewer incidents of misdiagnosis and inappropriate treatment.
Reduced loss of income due to illness.
Reduced stress

Further benefits are reduce costs due to earlier identification of the causes of dizziness in the care chain and hence facilitate fewer expensive specialist consultations, creation of efficiencies in the healthcare system by facilitating remote monitoring of patient's dizziness episodes and allow clinicians to see more patients in the same amount of time, reduction on the volume of ‘falls’ (and other outcomes that are the result of dizziness) due to earlier diagnosis of the causes of dizziness.

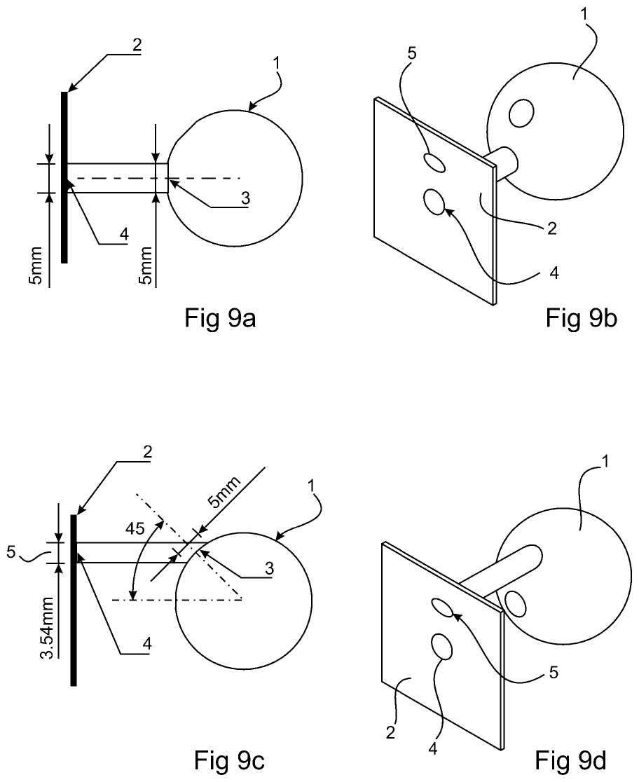

With reference to FIGS. 9A to 9D, the reference numerals indicate the eyeball 1, pupil projection onto capture apparatus image plane 2, actual pupil height 3, projected pupil height 4 and projected pupil shape 5.

In FIG. 9A the projection of the pupil image is 5 mm high, and the pupil projection is a circle with 5 mm diameter.

In FIG. 9C where the gaze (from eyeball 1) is 45 degrees upward, the pupil is still 5 mm however the pupil projection is a 3.54 mm high by 5 mm wide ellipse.

The change in shape of the ellipse projection of the pupil can be used to calculate the gaze angle and thereafter all related eye kinematics.

Information/data 30 directly or indirectly from the apparatus 10 of the present invention can be combined with sensed information/data from one or more head movement sensors 32 (such as one or more accelerometers), one or more body/body function sensors 34 and/or one or more external sensors 36, or a combination of any two or more thereof.

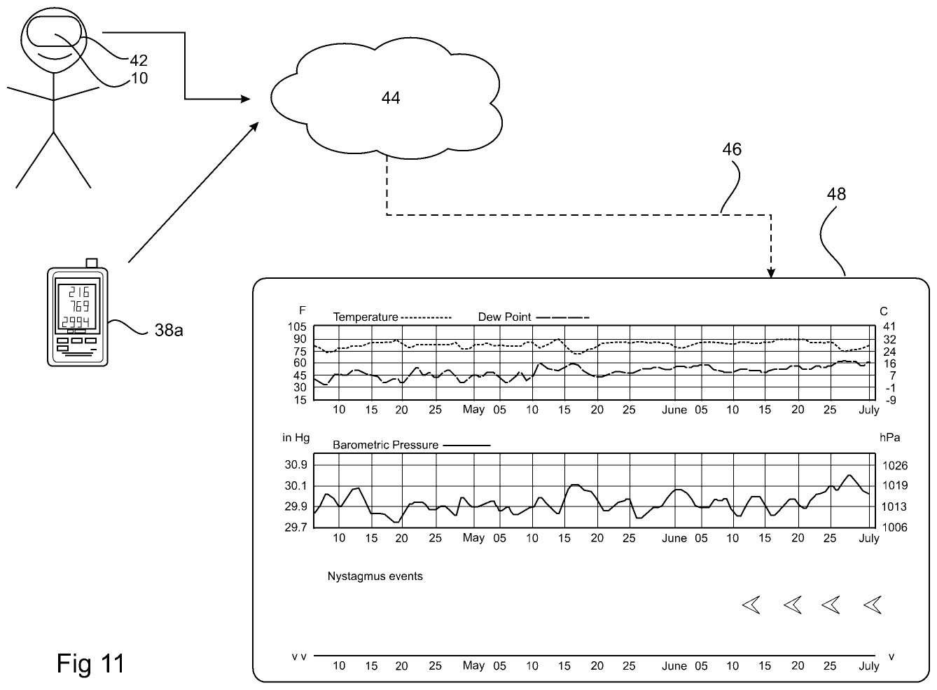

One or more external sensors 36 can include one or more environmental sensors 38a and/or one or more remote sensors 38b for remote monitoring of the person or their body factors e.g. remote cameras for capturing images for assessing behaviour, change in behaviour or activity (fitness, undertaking tasks, interaction with others etc.), temperature, blood pressure—see, for example, FIG. 11. For example, one or more environmental factors, such as temperature, air pressure, humidity, altitude, gravity, and/or rate of change of one or more thereof, can be measured. Measurement of the environmental factor(s) may be factored into assessing patient data, such as eye motion data 42 obtained via the apparatus 10. Analysis may be conducted using cloud computing/storage 44. Analysis in the cloud can examine and/or document relationship between environmental functions (such as temperature, air pressure (barometric pressure), humidity, gravity etc.) and the presence of eye motion or type of eye motion (such as nystagmus).

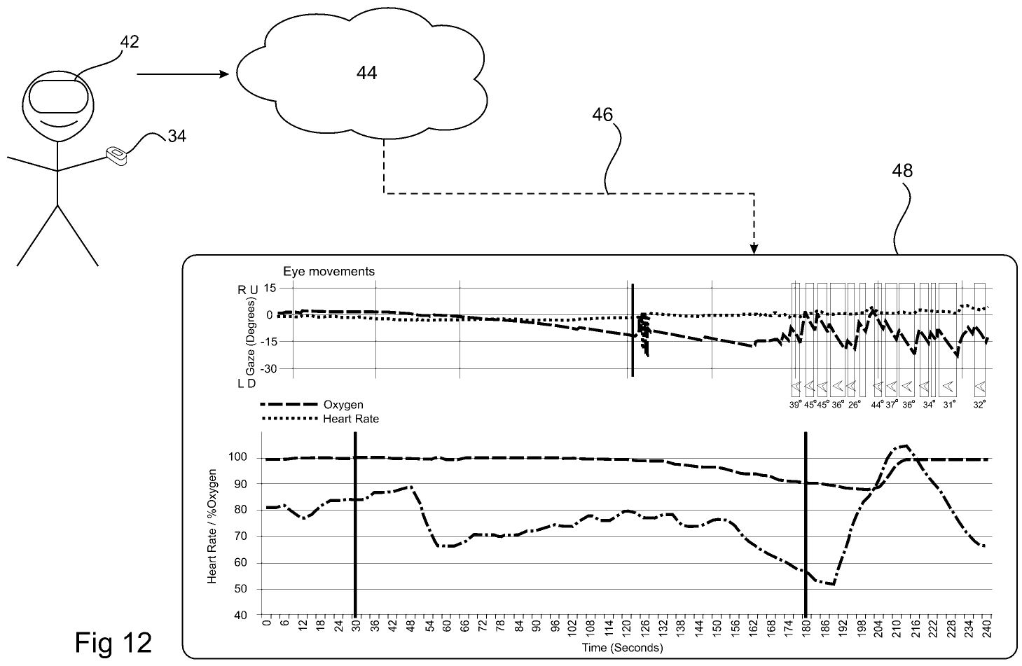

One or more body sensors 34 can include internal, body mounted or worn sensors, such as a heart rate monitor worn around the chest, a pulse sensor on the wrist, a blood pressure monitoring cuff on the arm. See, for example FIG. 12. For example, oxygen level and/or heart rate maybe measured and optionally provided as data for analysis with eye motion data 42 obtained using the apparatus 10. Eye motion data and the body measurement data can be provided via a cloud data service 44.

Analysis 46 can be conducted through cloud computing service. Such analysis may examine and/or document relationship between body functions (such as heart rate, oxygen level) and eye motion (such as nystagmus). Analysis/results can be displayed 48 for diagnosis/patent assessment.

Gravity (G) and/or changes in gravity (Gₜ) over time can be measured.

It will be appreciated that, whilst commensurate measurement of one or more of body, head and external parameters with eye state/movement is preferred, it can be possible to separately assess a person's physical parameters and assess gravity later or earlier.

Sensor information/data and/or information/data from the apparatus 10 can be provided for analysis by an analyser/processor 40.

Reaching or exceeding one or more threshold values of measured/sensed head metrics, body metrics and/or environmental metrics can be used to determine whether effect on eye state or change or eye state over time.

Head movement 50 and eye motion characteristics 52 can be measured. Accelerometer data 54 related to head movement can be provided as an input to understanding relationship between head movement and eye motion. Eye motion (such as a nystagmus pattern) can be detected 56, such as measurement of eye motion over time.

The eye motion pattern 58, such as indicating BPPV (benign paroxysmal positional vertigo) can be provided as part of the assessment of the relationship between head movement and eye motion—see 60.

Cause and effect relationship between specific head movement and specific eye motion can be assessed 60.

The specific otolith that is at fault can be assessed. For example, when an otolith is activated by specific head movement it causes an unwanted defect that is detectable or fails to cause an expected eye movement. By knowing the specific head movement and expected corresponding eye motion, the particular ear can be determined and the semi-circular canal within that ear can be identified that is causing the problem (See 62).

1. A method of assessing changes in eye state using an apparatus covering a user's eyes, wherein the apparatus detects or monitors changes in the eye(s) or movement of features of the eye(s) during a user vestibular or neurological episode or during a user vestibular or neurological event, the method including capturing image data of the user's eyes for a period of time during said episode or said event, and communicating the captured image data for assessing or informing diagnosis, absence or presence of a disorder or medical condition.

2. The method of claim 1, wherein a starting point and a finishing point of the pupil(s) is used to determine direction and magnitude of eye or pupil movement and/or other eye kinematics.

3. (canceled)

4. The method of claim 1, including capturing rotational motion of the eye(s) and/or pupil(s).

5. The method of claim 4, wherein time taken for a full or partial rotation is used to determine speed and/or rate of rotation and/or other eye kinematics.

6. The method of claim 1, wherein variation/change in angle and/or rate of change of such angle of the pupil(s) relative to a reference axis or point is captured as image data and/or measured.

7-9. (canceled)

10. The method of claim 1, including using pixel count of a sensor of a camera or each said camera of the apparatus to determine angle of gaze of the user, the method including determining angle of gaze from captured image data and/or image data recordings of eye/pupil position/movement.

11-12. (canceled)

13. The method of claim 1, including determining frame position by detecting that one or both eyes of the user of the apparatus is/are within a frame of reference covered by the at least one image capture/recording apparatus.

14. The method of claim 1, including identifying pupil position and/or pupil diameter in a reference region.

15. (canceled)

16. The method of claim 14, wherein a previsualization/detection field within which is captured the eye as the object of interest is used to ensure capturing the eye in image data and unnecessary frame/field beyond the eye is removed/reduced.

17. (canceled)

18. The method of claim 1, wherein a portion/window of an overall sensor area of the image capture apparatus is used for windowing such that captured image data is derived from the portion of the sensor area.

19. The method of claim 18, wherein the portion/window is moved to centralize with respect to the eye(s)/pupil(s) to frame the eye(s)/pupil(s) in at least one frame.

20. The method of claim 1, wherein position of the eye(s)/pupil(s) relative to one another and/or to a reference is used for positioning the apparatus on/relative to the user.

21. The method of claim 20, wherein height of a feature of one said eye relative to a feature of the other said eye and/or to at least one reference is used for positioning the apparatus on/relative to the user.

22. The method of or claim 21, wherein position of corners of the eyes are determined and used to compensate for position of the apparatus on the user's face.

23. The method of claim 1, wherein one or more of pupil center, pupil shape or pupil position relative to a horizontal plane/reference is used for calibration/position determination.

24. The method of claim 23, wherein, if the pupils are not aligned on a horizontal plane, degree of pupil misalignment is calculated and used to either realign eye images to a true horizontal plane or as an adjustment parameter in later image post processing and analysis.

25. The method of claim 1, including measuring/imaging variation in pupil shape from generally circular to ovoid/ellipsoid and compensating for non-circular irregularity via the image capture means and/or processing means.

26. The method of claim 25, including determining from the pupil shape a confidence factor in the accuracy of the eye kinematics.

27. The method of claim 26, wherein the confidence factor is used during analysis to modify or reject eye kinematic measurements.

28. The method of claim 26, wherein the confidence factor includes one or more of pixel count representing the shape of the pupil image, percentage of eyelid closure obstructing the view of the pupil, size of the pupil, angle of the pupil from the capture apparatus center.

29. The method of claim 1, including detecting full eye closure or partial eye closure occurring within a threshold time or occurring within a confidence factor of eye kinematics accuracy.

30-35. (canceled)

36. The method of claim 1, including detecting and/or imaging of presence of and/or location of at least one glint of one or both eyes relative to the center and/or edge of the respective pupil or iris.

37. (canceled)

38. The method of claim 36, wherein, for a given light source position within the apparatus, position of the glint is used to determine eye and/or pupil center position and/or wherein distance and/or position of the glint(s) from the respective pupil center is used to determine position and/or orientation of the apparatus or of a camera of the apparatus relative to the face of the user.

39. (canceled)

40. The method of claim 36, wherein distance between two said glints and/or between at least one said glint and the center of the pupil or other reference is used to compensate for non-ideal apparatus placement/orientation relative to the user's head/face with respect to eye position or to compensate for non-ideal positioning of at least one camera of the apparatus relative to eye position.

41-46. (canceled)

47. The method of claim 1, wherein one or more images or image data is used to identify the wearer or features of the wearer for personal identification, authentication, security, access control, authority to control/use equipment, authority to allow others such access/use.

48-77. (canceled)

78. A method of capturing and reporting eye motion data of a user for use in a medical diagnosis, or for producing a treatment regime, or for monitoring symptoms, for a user, the method including:
a portable eye motion capture apparatus self-administered by the user to cover their eyes prior to onset of, at the onset of, or during, or after, an episode or event of dizziness or imbalance
the eye motion capture apparatus configured for capturing eye motion data of the user during their episode or event of dizziness or imbalance
processing or part-processing the captured eye motion data onboard the portable eye motion capture apparatus or remote from the portable eye motion capture apparatus or a combination of onboard and remotely;
transmitting the respective processed, part processed or unprocessed eye motion data to a remote location creating analytics based on the processed, part processed or unprocessed eye motion data for use by a clinician in providing the medical diagnosis, or producing the treatment regime, or reporting on the symptoms, for said user.

79. The method of claim 78, including automatically detecting individual eye movements or patterns of eye movements within the processed and/or unprocessed said captured eye motion data.

80. The method of claim 79 including detecting nystagmus eye movements or patterns of nystagmus eye movements.

81. The method of claim 80, including determining speed and direction of the nystagmus eye movements and/or determining speed and direction of patterns of the nystagmus eye movements.

82. The method of claim 81, including displaying pattern, speed and/or direction of the nystagmus eye movements or patterns of eye movements as at least one graphic representation and/or on an electronic display.

83. The method of claim 81, including determining at least one driver or origin of the nystagmus from the pattern, speed or direction of the nystagmus.

84. The method of claim 80 including analyzing at least one component of the nystagmus or analyzing within phase eye kinematics of the nystagmus or analyzing at least one component of at least one slow phase of the nystagmus or analyzing transition(s) of one or more phases of the nystagmus or analyzing between nystagmus, or analyzing a combination of any two or more thereof, to determine drivers of the nystagmus, origin of the nystagmus and/or physiological pathways contributing to the eye movements.

85. The method of claim 78, wherein the eye motion data includes or is augmented by eye movement video sequences, 3D orientation and position of the user's head during their episode(s) or their event(s), eye kinematics and tracking data, other eye feature information, patient history, questionnaire data, information pertinent to diagnosis, or a combination of any two or more thereof.

86. The method of claim 78, including identifying severity, timing and location of the episode or event.

87. The method of claim 78, including instructing the user when and how to self-administer and use the eye motion capture apparatus.

88. The method of claim 78, wherein episode capture and/or recording is monitored in real or near real time by at least one clinician following automatic detection and transmission of recorded or direct image data as the episode or event occurs.

89. The method of claim 78, wherein the eye motion capture apparatus performs on-board processing that occurs during or after video capture of the eye motion data, wherein the on-board processing detects if the patient closes their eyes during the recording and/or after the data capture, compresses the video data and/or logs particular eye and head movements.

90. A method of remote oculography includes:
capturing nystagmus eye motion data of a user at their home, residence, place of work, or place of recreation, for use in a medical diagnosis, or for producing a treatment regime, or for monitoring symptoms, the method including:
a user self-administering a portable eye motion capture apparatus over their eyes prior to onset of, at the onset of, or during, or after, their vestibular or neurological episode or event;
the eye motion capture apparatus configured for capturing nystagmus eye motion data of the user during their episode or event;
processing or part-processing the captured nystagmus eye motion data onboard the portable eye motion capture apparatus, or remote from the portable eye motion capture apparatus, or a combination of onboard and remote;
transmitting the respective processed, part processed or unprocessed nystagmus eye motion data to a remote location;
creating analytics based on the processed, part processed or unprocessed nystagmus eye motion data for use by a clinician in providing the medical diagnosis, or producing the treatment regime, or reporting on the symptoms, for said user.
